(19) United States
(12) Patent Application Publication
(10) Pub. No.: US 2006/0046964 A1
(43) Pub. Date: Mar. 2, 2006

(54) Pharmaceutical formulations and methods

(76) Inventor: Andre Morneau, Aguadilla, PR (US)

Correspondence Address: DICKSTEIN SHAPIRO MORIN & OSHINSKY LLP, 2101 L Street, NW, Washington, DC, 20037, US

(21) Appl. No.: 11/216,042

(22) Filed: Sep. 1, 2005

Related U.S. Application Data
(60) Provisional application No. 60/606,525, filed on Sep. 2, 2004.

(51) Int. Cl. A61K 38/18; A61K 31/7012; A61K 31/715

(52) U.S. Cl. 514/12; 514/53; 514/59

(57) ABSTRACT

Stable sodium ferric carbohydrate complex intravenous formulations that are essentially free of sucrose or another disaccharide, methods of making such formulations, and methods for treating anemic conditions in a mammal by administering such compositions.

### CROSS-REFERENCE TO RELATED APPLICATIONS

This application claims the benefit of provisional application 60/606,525 filed Sep. 2, 2004, which is hereby incorporated by reference in its entirety.

### FIELD OF THE INVENTION

The field of this invention is sodium ferric carbohydrate pharmaceutical formulations, their use in treating various conditions (e.g., anemia), and methods of manufacturing such formulations.

### BACKGROUND OF THE INVENTION

Pharmaceutical formulations containing sodium ferric carbohydrate are indicated for the treatment of various conditions relating to iron deficiency. Iron is an essential nutrient required for tissue growth in humans and animals. While iron can be absorbed through food intake, the level of absorption is low and therefore may lead to iron deficiencies resulting from various conditions or diseases. For example, iron deficiency anemia may occur during pregnancy and may be present in newborns and young children. Disease conditions resulting in blood loss, improper distribution of iron, or loss of red blood cells may cause a state of chronic anemia. Such disease conditions can include, but are not limited to, Crohn's disease, rheumatoid arthritis, certain hemolytic diseases, and cancer.

Sodium ferric carbohydrate formulations (e.g., sodium ferric gluconate, iron-dextran, iron-sorbitol, and iron-poly(sorbitol-gluconic acid) are used, for example, to treat iron deficiency anemia. For example, sodium ferric gluconate complex in sucrose has been commercially available for treating iron deficiencies. See, e.g., U.S. Pat. No. 6,693,211. Sodium ferric gluconate complex, the active pharmaceutical ingredient of Ferrlecit® (Sodium Ferric Gluconate Complex in Sucrose Injection), is a macromolecular complex with an apparent molecular weight on gel permeability chromatography (GPC) of 289000 to 440000 Daltons.

Sodium ferric gluconate complex is indicated as first line treatment for iron deficiency anemia (depletion of total body content iron) in renal hemodialysis patients on supplemental recombinant human erythropoietin. Other treatments for anemia include Erythropoietin (EPO), which can be used to treat anemia associated with cancer, HIV infection, or AZT therapy. EPO is a kidney-secreted hormone that stimulates red blood cell production by bone marrow cells. EPO is available as a recombinant protein.

Parenteral administration of sodium ferric carbohydrate formulations significantly increases hemoglobin and transferrin levels as compared to iron administration via oral dosage forms. Nissenson et al., “Sodium ferric gluconate complex in sucrose is safe and effective in hemodialysis patients: North American clinical trial,” Am J Kidney Dis. 1999; 33(3):471-482.

Previously available sodium ferric carbohydrate formulations contain sucrose to help stabilize the sodium ferric carbohydrate complex. For example, Ferrlecit® contains 12.5 mg/ml of iron as the sodium salt of a ferric iron carbohydrate complex in alkaline aqueous solution, and further includes 195 mg/ml of sucrose. It has been theorized that the iron moiety of the complex is stabilized by surrounding it with sucrose and oxygen in a sucrose repeating unit linked to gluconate. It is believed that the sucrose molecules are coordinated to the iron atoms through a covalent bond between the iron and the alcohol linkage of the sucrose in a ratio of two iron to five sucrose molecules.

U.S. Pat. No. 6,537,820 (“the '820 patent”) refers to a method of monitoring iron saccharide complexes during manufacture. In particular, the '820 patent refers to “hematinic” compounds which comprise iron in a form that “tends to increase the amount of hemoglobin in the blood of a mammal, particularly in a human.” Col. 1, lines 16-19. All sodium ferric gluconate formulations referred to in the '820 patent include sucrose.

U.S. Pat. No. 6,693,211 (“the '211 patent”) refers to a process for preparing sodium ferric gluconate complex in sucrose. The process described in the '211 patent requires lyophilization of a sodium ferric gluconate complex and reconstitution of the complex in 20% sucrose at a temperature of between 80° C. and 100° C. Col. 3, lines 18-21.

Sterilization of a parenteral product is essential for the health and safety of patients and to comply with laws governing the manufacture and sale of drugs. Terminal sterilization is a procedure which subjects a final drug product to elevated temperature and pressure for a time sufficient to kill or inactivate microbes (e.g., bacteria, virus) in the final product. Other alternative methods of manufacturing sterile drug products include, for example, aseptic fill techniques.

In the absence of, or in addition to, sufficient sterilization, preservatives and other additives may be added to the formulation of drug products. Preservatives and additives may, however, preclude use of the drug in certain patient populations (e.g., children).

Inclusion of sucrose in sodium ferric carbohydrate complex formulations limits the options available for sterilization of the final product. Terminal sterilization can reduce the yield and effectiveness of the active ingredient in sucrose-containing products. Upon terminal sterilization, the iron(III) content of the sodium ferric carbohydrate complex in sucrose formulations decreases by 5 to 10% depending on the duration of the sterilization process. Also, a change of appearance and color of the sodium ferric carbohydrate complex in sucrose formulations is apparent after the sterilization. As a result, sucrose-containing products are aseptically filled or lyophilized with a reconstitution step prior to use. The use of aseptic fill or lyophilization procedures gives rise to a risk of contamination during the fill procedure or during the reconstitution step. What is needed are stable, ready-to-use, sodium ferric carbohydrate formulations which are capable of being sterilized by a sterilization technique, such as, for example terminal sterilization.

### BRIEF DESCRIPTION OF THE DRAWINGS

FIG. 1 shows the molecular weight profile of a sodium ferric carbohydrate complex composition according to an embodiment of the invention which has not been terminally sterilized.

FIG. 2 shows the molecular weight profile of a sodium ferric carbohydrate complex composition according to an embodiment of the invention which has been terminally sterilized.

FIG. 3 depicts a proposed structure of a sodium ferric carbohydrate complex in accordance with a preferred embodiment of the invention.

FIG. 4 shows the iron-57 Mössbauer Spectroscopy analysis of sodium ferric carbohydrate complex formulations with and without sucrose.

FIG. 5 shows the results of stability tests conducted on sodium ferric carbohydrate complex without sucrose formulations according to another embodiment of the present invention.

### SUMMARY OF THE INVENTION

We have discovered that sucrose-free sodium ferric carbohydrate complex pharmaceutical formulations are stable, and retain their stability even after terminal sterilization. Thus, it is no longer necessary to include sucrose in the manufacture of sodium ferric carbohydrate complex pharmaceutical formulations in order to achieve sufficient yield and stability of the iron complex.

Terminal sterilization of previous sucrose-containing intravenous formulations of sodium ferric carbohydrate complex results in insufficient yield and stability of the iron complex. In order to obtain a sterile sucrose-containing formulation, the sodium ferric carbohydrate complex is lyophilized and reconstituted in sterile saline prior to use. Reconstituting formulations prior to use is inconvenient and may expose patients to the risk of error on the part of medical staff. Sodium ferric gluconate complex in sucrose cannot be terminally sterilized without a loss of potency of iron(III) in the formulation. Furthermore, lack of terminal sterilization may require the addition of a preservative to prevent microbial contamination, which in turn limits the patient population that may receive the drug. For example, pediatric indications may not be available for drugs which contain the preservative benzyl alcohol.

Preferred embodiments of the invention provide pharmaceutical compositions comprising a therapeutically effective amount of a sodium ferric carbohydrate complex, a salt, and water wherein the composition is sucrose-free or essentially sucrose-free. In another embodiment, the pharmaceutical compositions are disaccharide-free (e.g., maltose, lactose, trehalose, ribose, and fructose) or essentially disaccharide-free. The carbohydrate can be any suitable carbohydrate (such as, for example, a linear carbohydrate). For example, the carbohydrate can have from 2 to 8 carbons. In one embodiment, the carbohydrate is a polyhydroxy acid (e.g., gluconic acid, gulonic acid, glucoheptonic acid, and lactic acid). The salt (e.g., sodium chloride, sodium succinate) can be present in the composition, for example, in a concentration of from about 5 mg/ml to about 11 mg/ml, preferably about 9 mg/ml. Preferably, the composition is essentially preservative-free. The concentration of iron in the composition is typically from about 0.5 mg/ml to about 12.5 mg/ml, preferably about 1.25 mg/ml. In one embodiment, the pH of the composition can be from about 6 to about 10, preferably about 7. The composition is preferably isotonic with an osmolarity from about 200 mOsm/L to about 400 mOsm/L, most preferably about 300 mOsmol/L.

Preferred embodiments of the invention also provide methods for preparing a parenteral pharmaceutical composition comprising dissolving sodium chloride in water to form a sodium chloride solution, adding sodium ferric carbohydrate complex to the sodium chloride aqueous solution, thus forming a sodium ferric carbohydrate complex solution, and, if desired, subjecting the sodium ferric carbohydrate solution to terminal sterilization.

In yet another embodiment, terminal sterilization comprises subjecting the pharmaceutical composition to a suitable temperature and pressure for a period of time sufficient to sterilize the pharmaceutical composition, preferably a temperature from about 115° C. to about 125° C., a pressure from about 10 to 20 PSI, and a period of time from about 15 to 60 minutes, although various other temperatures, pressures, and time periods may be used. Additionally, the composition can be sterilized by aseptic filling, gamma irradiation or with treatment by ethylene oxide.

The invention also provides methods for preparing a sodium ferric carbohydrate complex pharmaceutical formulation comprising collecting water at about 80° C., degassing the water with nitrogen, cooling the water to about 25° C., admixing sodium chloride to a concentration of about 9 mg/ml, admixing sodium ferric carbohydrate complex to a concentration of about 1.25 mg/ml of iron, and subjecting the formulation to terminal sterilization, wherein the pH of the resulting composition is about 7 and the osmolarity is about 300 mOsmol/liter.

Further embodiments of the invention include methods of treating anemic conditions in a mammal comprising parenterally administering an anti-anemic effective amount of a sodium ferric carbohydrate pharmaceutical formulation which is free of sucrose or essentially sucrose free. In another embodiment, the anti-anemic effective amount of a sodium ferric pharmaceutical formulation is free of disaccharide or essentially disaccharide-free.

Additional features and advantages of the invention are set forth in the description which follows and will be apparent from the description or may be learned by practice of the invention.

### DETAILED DESCRIPTION OF THE INVENTION

Reference will now be made in detail to the presently preferred embodiments of the invention, which, together with the following examples, serve to explain the principles of the invention. It is to be understood that the application of the teachings of the present invention to a specific problem or environment will be within the capabilities of one having ordinary skill in the art in light of the teachings contained herein.

The present invention provides stable pharmaceutical compositions which are aqueous solutions comprising a therapeutically effective amount of a sodium ferric carbohydrate complex. As used herein, “stable” means that the characteristics of the pharmaceutical compositions remain within pharmaceutically acceptable guidelines over the desired shelf life, typically at least about two months, six months, one year, eighteen months, two years, three years, or five years. Such guidelines meet or exceed the standards for a drug product to be administered to a human or animal in various jurisdictions (e.g., Australia, U.S., Europe, Japan, Canada, etc.).

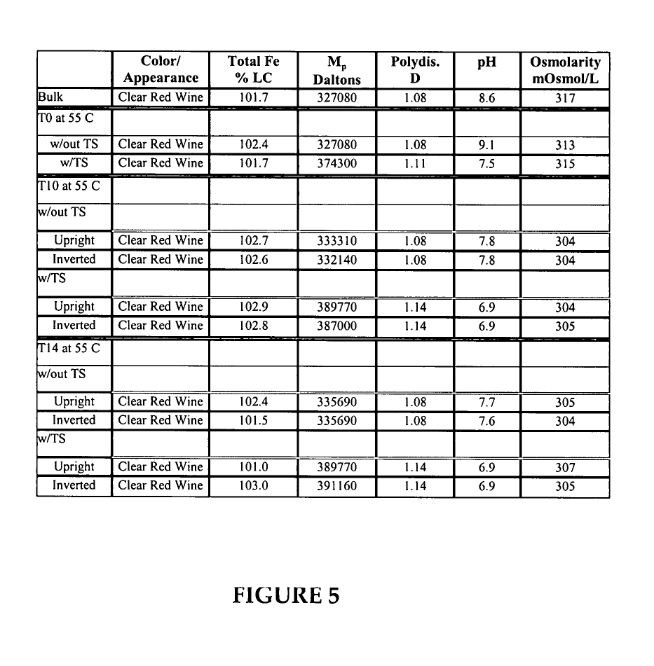

Various characteristics of the compositions are indicative of “stability” as defined herein. In one embodiment, the concentration of iron in compositions of the invention remains substantially the same for the desired shelf life. The concentration of iron, the active ingredient in the sodium ferric carbohydrate complex, affects the potency of the drug product. In another embodiment, the pH of compositions of the invention also remains substantially the same over a desired shelf life. Preferably, the pH of the compositions are substantially the same pH as the blood. In one embodiment, the pH of the compositions are isohydric. The osmolarity of the preferred compositions of the invention also remains substantially the same over at least a desired shelf life, in accordance with another embodiment of the invention. FIG. 5 provides exemplary data showing various stability characteristics of preferred embodiments of formulations in accordance with the present invention, but other embodiments are also stable as indicated by the discussion above.

In addition, a change of color or the appearance of particulate matter in compositions can indicate a lack of the appearance relative stability of sodium ferric carbohydrate complex pharmaceutical formulations. In an embodiment of the invention, the color and lack of particulate matter in the pharmaceutical compositions remains substantially the same for at least a desired shelf life, (e.g., preferably at least two months, six months, one year, eighteen months, two years, three years, or five years), or after 14 days of accelerated stability testing at 55° C.

In a particularly preferred embodiment of the invention, the concentration of iron, the pH, and the osmolarity of the compositions of the invention remain substantially the same over at least a desired shelf life. See FIG. 5, for example. In an especially preferred embodiment, the iron concentration of about 1.25 mg/ml, pH of about 7.0 to about 7.5, and osmolarity of about 310 mOsmol/L remain the same over the shelf life.

The preferred compositions of the present invention, are said to be free or “essentially free” of sucrose. In another embodiment the preferred compositions of the invention are free or “essentially free” of a another disaccharide. The term “essentially free of sucrose” or “essentially-free of disaccharide” refers to trace amounts or higher levels of sucrose or another disaccharide (e.g., maltose, lactose, trehalose, ribose, and fructose) that are sufficiently low so as not to substantially reduce the stability of the pharmaceutical composition following terminal sterilization. Pharmaceutical compositions of the invention also include compositions that are free or essentially free of sucrose or another disaccharide that are not terminally sterilized.

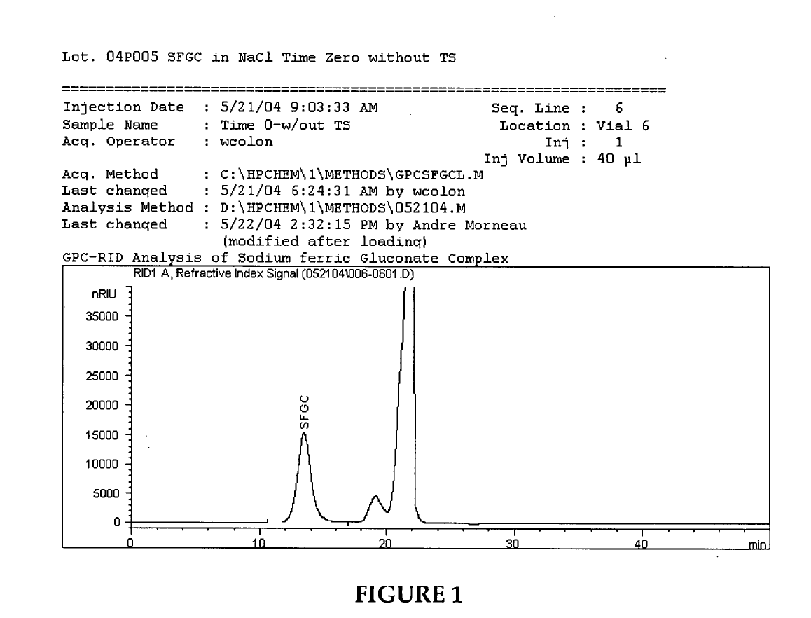

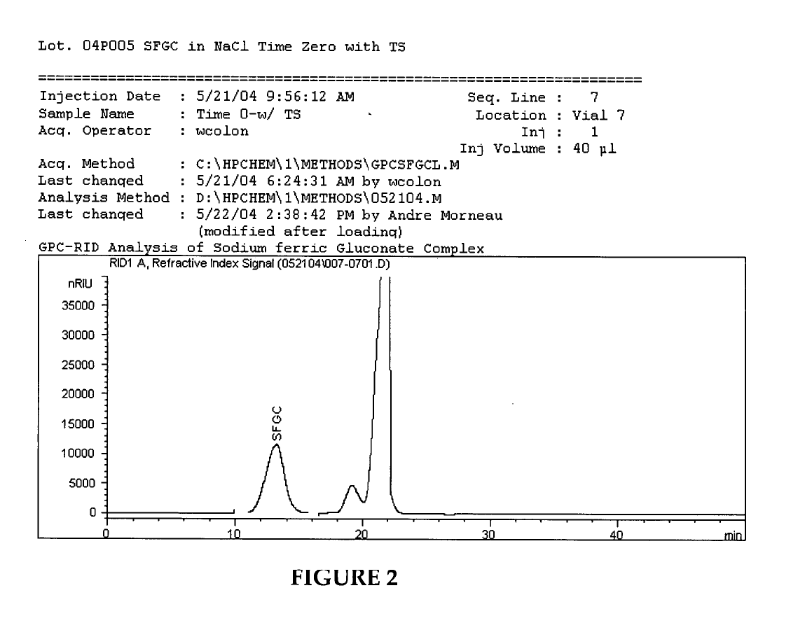

The compositions of the invention have a substantially constant molecular weight profile before and after terminal sterilization as shown in FIGS. 1 and 2. FIG. 1 shows the molecular weight profile of a preferred composition of the invention containing sodium ferric carbohydrate complex, sodium chloride, and water without terminal sterilization. Gel permeation chromatography using a refractive index detector (GPC-RID) of the samples was performed at 1.25 mg/mL of iron and shows a sodium ferric carbohydrate complex peak at an elution time 13.494 minutes and an apparent molecular weight of 327080 Daltons. FIG. 2 shows the molecular weight profile of the same composition after terminal sterilization with a sodium ferric carbohydrate complex peak at an elution time of 13.216 minutes and an apparent molecular weight of 374300 Daltons. Thus, the molecular weight of the sodium ferric carbohydrate complex in the preferred compositions of the invention is substantially the same before and after terminal sterilization.

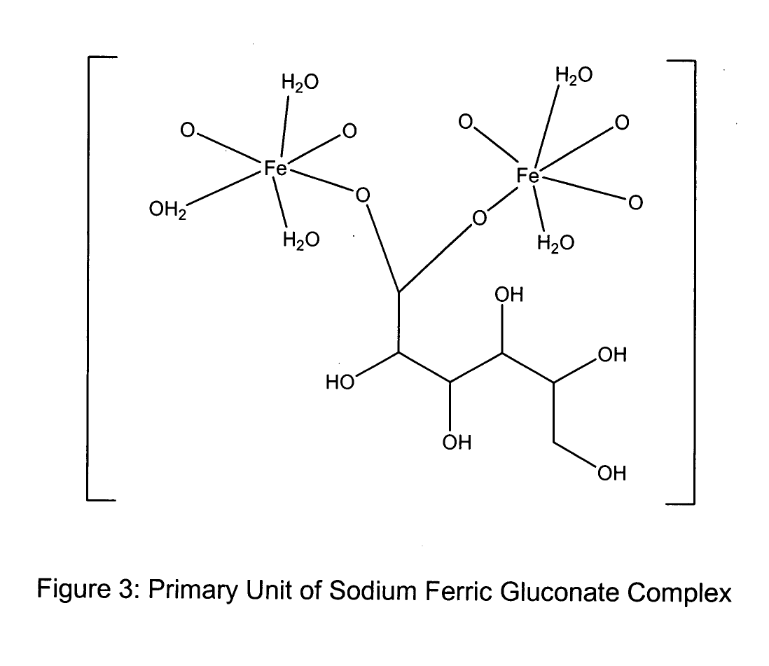

While not wishing to be bound by theory, it is believed that the structure of a primary repeating unit of the macromolecular sodium ferric gluconate complex in accordance with an embodiment of the invention is as depicted in FIG. 3. The structure depicts two different oxygen-ligated iron atoms, or “iron centers” making up part of the sodium ferric carbohydrate complex. The structure of the first iron center is composed of an iron atom coordinated to one oxygen from the carboxyl functionality of the gluconate moiety, two oxygen atoms, which are bridged to another iron center of the next repeating unit of the macromolecule, and three oxygen atoms associated with the solvated water molecules. The second iron center is composed of an iron atom coordinated to the remaining oxygen of the carboxy functionality of the gluconate moiety, three oxygen atoms which are bridged to another iron center of the next repeating unit of the macromolecule, and two oxygen atoms associated with the solvated water molecules. The first and second iron centers can also be expressed as: Fe(O_{Glu})(Oₒₓₒ)₂(O_{water})₃ and Fe(O_{Glu})(Oₒₓₒ)₃(O_{water})₂ respectively. Because of the asymmetric ligand configurations, two coordinated iron species exist, each possessing different Mössbauer spectrographic characteristics.

Mössbauer Spectroscopy is a spectroscopic technique used to analyze the properties of materials such as iron-57 (or Fe⁵⁷). Using this technique, a solid or a frozen liquid sample is exposed to a beam of gamma radiation, and a detector measures the intensity of the beam that is transmitted through the sample. The resulting gamma spectra provides information about the chemical environment of the absorbing nuclei and can be used to characterize the sample.

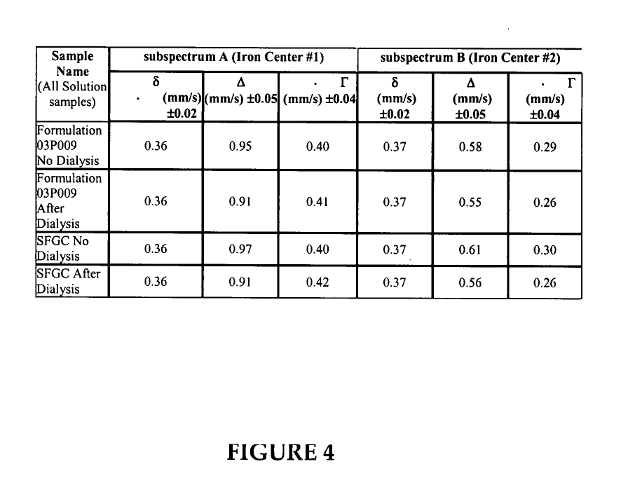

Iron-57 Mössbauer Spectroscopy analysis of sodium ferric carbohydrate complex formulations with or without sucrose is shown in FIG. 4. Based on these iron-57 Mössbauer results, we have discovered that the presence of sucrose does not affect the iron atoms of the sodium ferric carbohydrate complex. The iron (Fe) atoms of the sodium ferric carbohydrate complex are therapeutically beneficial to patients suffering from conditions such as anemia. Surprisingly, the presence or absence of sucrose in sodium ferric carbohydrate complex formulations has little to no effect on the iron atoms.

Mössbauer analysis also was performed on a non-terminally sterilized sodium ferric carbohydrate complex formulation, having a concentration of approximately 12.5 mg/mL of iron. As shown in FIG. 4, the iron in the complex has a characteristic isomer shift (approximately 0.35 mm/s, relative to metal iron). This isomer shift is consistent with the known ferric oxidation state for Fe⁺³.

The sodium ferric carbohydrate complex formulations of the invention advantageously remain stable before and after terminal sterilization. As shown in FIG. 5, formulations of the invention were terminally sterilized (w/TS) or not terminally sterilized (w/out TS). These formulations were incubated at 55° C. and tested for stability at 0, 3, 7, 10, and 14 days. The color/appearance, total ion concentration, molecular weight, polydispersion, pH, and osmolarity remained substantially constant over this period of time. Thus, terminal sterilization does not have an adverse impact on the characteristics of the preferred compositions of the invention.

The formulations of the present invention can be prepared by various methods. One embodiment of a manufacturing method is as follows.

### Manufacture of Sodium Ferric Gluconate Intravenous Liquid Pharmaceutical Formulation

Exemplary Formula:

| Item# | | Quantity per 1 mL |
|---|---|---|
| 1 | Sodium Ferric Gluconate Complex (Iron) | 1.25 mg |
| 2 | Sodium Chloride, USP | 9 mg |
| 3 | Water for Injection (WFI), USP | q.s. to 1.0 mL |

Exemplary Process of Making Intravenous Formulation of Sodium Ferric Gluconate Complex

| Step | Description |
|---|---|
| A | Collection of WFI (80° C.) into the formulation tank. |
| B | Apply Nitrogen blanket and sparging to formulation tank. |
| C | Transfer WFI into a pressure vessel. Keep nitrogen pressure |
| | in the pressure vessel at not more than 15 psig. |
| D | Adjust the mixer air pressure and resume mixing at a speed |
| | of 630 ± 30 rpm in the formulation tank. Continue nitrogen |
| | blanketing and sparging throughout the process at a flow rate |
| | of 1.5 ± 0.5 SCF/M. Maintain the temperature at 25 ± 5° C. |
| | during the formulation process. |
| E | While mixing at approximately 630 ± 30 rpm, add 630 g |
| | of Sodium Chloride, USP into the formulation tank. Continue |
| | mixing at the same speed for 5-10 minutes before proceeding |
| | with the next step. |
| F | Stop nitrogen blanket. While mixing at approximately |
| | 630 ± 30 rpm, slowly add 292 g of Sodium Ferric Gluconate |
| | Complex into the formulation tank as close to solution as |
| | possible in order to avoid airborne dust. Resume nitrogen |
| | blanket and sparging at a flow rate of 2.0 ± 0.5 SCF/M. |
| | Continue mixing at the same speed for 15-20 minutes before |
| | proceeding to Step 5.11. Verify the temperature of the solution |
| | using the Resistance Thermometer Device or thermometer. |
| G | Optionally, the resulting sodium ferric carbohydrate solution |
| | can be aseptically filtered through a 0.22 μm filter. |
| H | Apply terminal sterilization procedure at 115° C. to about |
| | 125° C., with pressure between 10 and PSI from about 15 |
| | to about 60 min. |

In another preferred embodiment, formulations of the invention can be prepared by collecting water, degassing the water with nitrogen, adding about 10-80 grams of sodium chloride at a concentration of about 5-15 mg/ml, adding about 12-45 grams of sodium ferric gluconate complex with a concentration of about 1.25 to about 12.5 mg/ml of iron, adding water until the total volume is about six liters, and subjecting the pharmaceutical composition to terminal sterilization wherein the pH of pharmaceutical composition after terminal sterilization is about 6-8 and the osmolarity of the pharmaceutical composition is about 100-500 mOsmol/liter.

The carbohydrate component of the sodium ferric carbohydrate complex can be any suitable carbohydrate that provides for a stable macromolecular complex. For example, linear carbohydrates having from 2 to 8 carbons can be used to provide the carbohydrate component of the complex. Polyhydric acids (e.g., lactic acid, and gluconic acid) can also provide a suitable carbohydrate component for the complex. In one preferred embodiment, gluconate is the carbohydrate component of the sodium ferric carbohydrate complex.

Sodium ferric carbohydrate complex without sucrose can be obtained from a suitable source (e.g., Cilag AG, Hochster 201, CH-8205 Schaffhaufen, Switzerland). In one embodiment, gluconate is the carbohydrate used to form the complex although other suitable carbohydrates can be used. The compositions are preferably free of or essentially free of sucrose and remain stable following terminal sterilization.

Any pharmaceutically acceptable salt can be used in the manufacture of the sodium ferric carbohydrate complex formulations of the invention. The pharmaceutically acceptable salt (e.g., sodium chloride) is preferably present in an amount sufficient to result in an isotonic composition. The pharmaceutically acceptable salt will typically be present in a concentration of about 5 mg/ml to about 11 mg/ml, preferably in a concentration of about 9 mg/ml.

In one embodiment, the pH of the compositions of the invention are from about 6 to about 10. In another preferred embodiment, the pH of the terminally sterilized product is about neutral and preferably in the range (7.30-7.35), which is typically the pH of blood. In one embodiment, the pH of the composition prior to terminal sterilization is in the alkaline range (e.g., 8-9). After terminal sterilization the pH can be, for example, in the neutral range (e.g., 6-8).

The compositions of the invention are preferably preservative-free. Benzyl alcohol, a preservative, is potentially toxic to neonates. See, e.g., Gershanik et al., The Gasping Syndrome And Benzyl Alcohol Poisoning, New England Journal of Medicine, 207:1384-1388 (1982). The use of terminal sterilization, which is permitted by virtue of the essentially sucrose-free characteristic of the formulations, eliminates the need for inclusion of a preservative.

Preferably, the compositions of the invention are isotonic. In one embodiment, the composition has an osmolarity of about 200 mOsm/L to about 400 mOsm/L. In a preferred embodiment, the osmolarity is about 270-330 mOsm/L.

After manufacture, the formulations of the invention may be packaged in any suitable container for use by health care providers and patients. Suitable packaging includes bags, various glass, PET, and HDPE bottles, preferably opaque HDPE to further enhance long term stability. Formulations of the invention can also be provided in ready-to-use bags for immediate administration to patients, since the formulation is terminally sterilized and does not need to be reconstituted prior to use.

The present invention also provides methods of treating anemic conditions. Anemic conditions can be caused by a variety of diseases including cancer, kidney disease, and HIV infection. Anemia may also result from treatments for disease. For example, anemia is a side effect of certain chemotherapies.

Methods of treatment according to the invention include administering to a mammal (e.g., human) suffering from an anemic condition a pharmaceutical composition according to the invention. For example, administration may be by various routes including but not limited to oral, subcutaneous, intravenous, intradermal, intramuscular, and intraperitoneal. Parenteral administration can be by bolus injection or by gradual perfusion over time. It is understood that the dosage will be dependent upon the age, sex and weight of the recipient, kind of concurrent treatment, if any, condition being treated, frequency of treatment, and the nature of the effect desired. The ranges of effective doses provided below are not intended to limit the invention and merely represent preferred dose ranges. However, the most preferred dosage will be tailored to the individual subject as is understood and determinable by one skilled in the art. The total dose required for each treatment may be administered by multiple doses over a period of time or in a single dose. In one embodiment, effective amounts of composition have an iron concentration from about 0.01 mg/ml to about 100 mg/ml, and preferably from about 0.5 mg to about 12.5 mg/ml.

The compositions of the invention may be administered alone or in conjunction with other therapeutics directed to the same or other conditions. For example, treatment with compositions of the invention can precede, follow, or be conducted concurrently with treatment with EPO. In one embodiment, an effective amount of EPO is an amount of EPO sufficient to have an anti-anemic effect in a mammal. Treatment with compositions of the invention may precede or follow treatment with EPO by multiple intervals ranging from minutes to weeks. In another embodiment, compositions of the invention and EPO are administered such that a prolonged period of time does not elapse between the time of administration of each agent. For example, each composition can be administered to a patient within seconds, minutes, or hours of the other composition.

Any suitable regimen for administration of compositions according to the invention can be used. For example, 125 mg of iron in compositions of the invention having a concentration of 19 mg/L can be administered in 7 minutes. Alternatively, compositions of the invention can be administered at a rate of 62.5 mg in five minutes.

It is to be understood that application of the teachings of the present invention to a specific problem or environment will be within the capability of one having ordinary skill in the art in light of the teachings contained herein. The present invention is more fully illustrated by the following non-limiting example.

### EXAMPLE 1

The following is an exemplary method of making a sodium ferric gluconate complex intravenous formulation in accordance with a preferred embodiment of the invention:

Add approximately 70 Kg of hot water (WFI (80±5 C)) to a formulation tank and record the gross weight.

Set-up a nitrogen blanket and sparging lines on the formulation tank. For sparging, connect one end of a clean nitrogen line hose to the flow meter located on the nitrogen manifold and the other end to the quick-connector located on the shoulder of the formulation tank. For blanketing, connect one end of a clean nitrogen line hose to the flow meter located on the nitrogen manifold and the other end to the other quick-connector located on the shoulder of the formulation tank. Blanket and sparge with nitrogen, NF at 1.5±0.5 SCF/M throughout the formulation process.

Adjust the air pressure on the mixer in the formulation tank to achieve a mixing speed of 630±30 rpm and start mixing. Cool and maintain the WFI in the formulation tank to a temperature of 25±5° C. by circulating water from the chiller or heat exchanger throughout the tank's jacket.

Measure the dissolved oxygen content in the formulation water using a K-7501 Chemets Kit. The dissolved oxygen content must be not more than 0.5 PPM. If the dissolved oxygen content is more than 0.5 PPM, continue nitrogen sparging and blanketing in 15-minute intervals until the oxygen content is not more than 0.5 PPM

Stop mixing and empty the tank's jacket by applying compressed air at approximately 15 psi. Remove the air hose from the jacket and record the amount of WFI in the tank measuring it by weight.

Transfer approximately 20 Kg of WFI from the formulation tank into a 45-L pressure vessel. The transferred WFI in the 45-L pressure vessel is to be used for container rinses, final volume adjustment and bubble point test. Keep nitrogen pressure in the 45-L pressure vessel at not more than 15 psig. Using a weighing scale, verify water remaining in the formulation tank.

Adjust the mixer air pressure and resume mixing at a speed of 630±30 rpm in the formulation tank. Continue nitrogen blanketing and sparging throughout the process at a flow rate of 1.5±0.5 SCF/M. Maintain the temperature at 25±5° C. during the formulation process.

While mixing at approximately 630±30 rpm, add 630 g of Sodium Chloride, USP into the formulation tank. Record the time of addition. Rinse each container with 100 mL of WFI from the 45-L pressure vessel and pour the rinse solution back into the formulation tank. Continue mixing at the same speed for 5-10 minutes before proceeding with the next step. Verify the temperature of the solution using the resistance temperature detector or thermometer.

Stop nitrogen blanket. While mixing at approximately 630±30 rpm, slowly add 292 g of Sodium Ferric Gluconate Complex into the formulation tank as close to solution as possible in order to avoid airborne dust. Record the time of addition. Rinse the container with 100 mL of WFI from the 45-L pressure vessel and pour the rinse solution back into the formulation tank. Resume nitrogen blanket and sparging at a flow rate of 1.5±0.5 SCF/M. Continue mixing at the same speed for 15-20 minutes.

Stop mixing and sparging. Allow the solution to settle for one (1) minute. Collect approximately 4 liters of solution into a clean 6-L depyrogenated glass flask for dissolution check. From the 4 liters of solution, place 100 mL in a 1-L flask and check for undissolved particles.

If the solution in the flask contains undissolved materials, pour the solution back into the formulation tank. Adjust the mixer air pressure and resume mixing at a speed of 630±30 rpm and sparging at 1.5±0.5 SCF/M for ten to fifteen (10-15) minutes and repeat dissolution check as needed. Once the solution in the flask is free of undissolved materials, pour the solution back into the formulation tank and proceed to the next step.

Stop nitrogen blanketing in the formulation tank. Make sure that the tank's jacket is empty by applying compressed air at approximately 15 psi. Remove the air hose from the tank, and the tank from the scale. Verify that the scale weight reads zero, if not, reset and replace the tank on the scale. Accurately adjust the final volume to 72.2 Kg using the weighing scale, by carefully adding WFI from the 45-L pressure vessel reserved to the formulation tank. Resume sparging and blanketing at a flow rate of 1.5±0.5 SCF/M. Adjust mixer air pressure and resume mixing at 630±30 rpm for 10 to 15 minutes.

Calibrate the pH meter with pH 7, 10 and pH 12 standard solutions to a pH of 7. Determine and record the pH of the solution in the formulation tank. The pH of the solution prior to terminal sterilization should be in the range of 9.0 to 10.2. Terminally sterilize the solution, for example, by subjecting the solution to a temperature from about 115° C. to about 125° C., a pressure from about 10 to 20 PSI, and a period of time from about 15 to 60 minutes. After terminal sterilization, the pH of the solution can be in the range of about 6.5 to 7.5.

The above description and example are only illustrative of preferred embodiments which achieve the objects, features, and advantages of the present invention, and it is not intended that the present invention be limited thereto.

What is claimed as new and desired to be protected by Letters Patent of the United States is:

1. A pharmaceutical composition, comprising a therapeutically effective amount of a stable sodium ferric carbohydrate complex and water, wherein the composition is essentially sucrose-free.

2. The pharmaceutical composition of claim 1, wherein said composition is sucrose-free.

3. The pharmaceutical composition of claim 1, wherein said composition is suitable for parenteral administration.

4. The pharmaceutical composition of claim 1, wherein said composition is suitable for intravenous administration.

5. The pharmaceutical composition of claim 1, wherein the carbohydrate is polyhydroxy acid.

6. The pharmaceutical composition of claim 5, wherein the polyhydroxy acid is selected from the group consisting of gluconic acid, gulonic acid, glucoheptonic acid, and lactic acid.

7. The pharmaceutical composition of claim 1, wherein the salt is sodium chloride present in a concentration of from about 5 mg/ml to about 11 mg/ml.

8. The pharmaceutical composition of claim 7, wherein the sodium chloride is present in a concentration of about 9 mg/ml.

9. The pharmaceutical composition of claim 1, wherein the pH of the composition is from about 6 to about 10.

10. The pharmaceutical composition of claim 1, wherein said composition is essentially preservative-free.

11. The pharmaceutical composition of claim 1, wherein the composition has an osmolarity of about 150 mOsm/L to about 400 mOsm/L.

12. The pharmaceutical composition of claim 11, wherein the composition has an osmolarity of about 300 mOsm/L.

13. The pharmaceutical composition of claim 12, wherein the composition is isotonic.

14. A stable pharmaceutical composition, consisting essentially of iron as the sodium salt of a ferric ion carbohydrate complex, sodium chloride, and water.

15. The composition of claim 14, wherein said composition has a pH of about 7, and an osmolarity of 300 mOsmol/L.

16. A stable, intravenous pharmaceutical composition, consisting essentially of about 1.25 mg/ml of iron as the sodium salt of a ferric ion carbohydrate complex, about 9 mg/ml of sodium chloride, and water wherein the pH of the pharmaceutical composition is about 7, and the osmolarity of the pharmaceutical composition is about 300 mOsmol/L.

17. A pharmaceutical composition, comprising a therapeutically effective amount of a stable sodium ferric carbohydrate complex, a pharmaceutically acceptable salt, and water, wherein the composition is essentially disaccharide-free.

18. A method for preparing a stable pharmaceutical composition, comprising:
dissolving sodium chloride in water forming a sodium chloride solution;
admixing sodium ferric carbohydrate complex with the sodium chloride aqueous solution to form a sodium ferric carbohydrate complex solution;
and subjecting the sodium ferric carbohydrate complex solution to terminal sterilization;
wherein the sodium ferric carbohydrate complex is essentially sucrose-free.

19. The method of claim 18, wherein the temperature for the terminal sterilization is from about 115° C. to about 125° C.

20. The method of claim 18, wherein the pressure for the terminal sterilization is from about 10 to 20 PSI.

21. The method of claim 18, wherein the period of time for the terminal sterilization is from about 15 to 60 minutes.

22. A method for preparing a stable pharmaceutical composition, comprising:
dissolving sodium chloride in water forming a sodium chloride solution;
admixing sodium ferric carbohydrate complex with the sodium chloride aqueous solution to form a sodium ferric carbohydrate complex solution;
sterilizing the sodium ferric carbohydrate complex solution;
wherein the sodium ferric carbohydrate complex is essentially sucrose-free.

23. The method of claim 22, wherein said sodium ferric carbohydrate complex solution is sterilized by aseptic filling.

24. The method of claim 22, wherein said sodium ferric carbohydrate complex solution is sterilized by terminal sterilization.

25. The method of claim 22, wherein said sodium ferric carbohydrate complex solution is sterilized by gamma irradiation.

26. The method of claim 22, wherein said sodium ferric carbohydrate complex solution is sterilized with ethylene oxide.

27. A method for preparing the pharmaceutical composition of claim 1, comprising the steps of collecting water, degassing the water with nitrogen, adding about 10-80 grams of sodium chloride at a concentration of about 5-15 mg/ml, adding about 12-45 grams of sodium ferric gluconate complex with a concentration of about 1.25 to about 12.5 mg/ml of iron, adding water until the total volume is about six liters, and subjecting the pharmaceutical composition to terminal sterilization wherein the pH of pharmaceutical composition after terminal sterilization is about 6-8 and the osmolarity of the pharmaceutical composition is about 100-500 mOsmol/liter.

28. A method of treating conditions in a mammal comprising administering to said mammal a therapeutically effective amount of a pharmaceutical composition as defined in claim 1.

29. A method of treating anemic conditions in a mammal comprising administering to said mammal an anti-anemic effective amount of a pharmaceutical composition as defined in claim 1.

30. The method of claim 27, wherein the mammal is a human.

31. The method of claim 27, wherein the administration is intravenous.

32. A method of treating anemic conditions in a mammal comprising administering to said mammal an anti-anemic effective amount of a pharmaceutical composition as defined in claim 1 and an effective amount of erythropoietin.
